(19) United States
(12) Patent Application Publication
(10) Pub. No.: US 2007/0060933 A1
(43) Pub. Date: Mar. 15, 2007

(54) Curette heads

(76) Inventors: Meera Sankaran, Cupertino, CA (US); Alberto Ruiz Cantu, San Francisco, CA (US); Richard W. Layne, Phoenix, AZ (US)

Correspondence Address: FISH & RICHARDSON P.C., PO BOX 1022, MINNEAPOLIS, MN, 55440-1022, US

(21) Appl. No.: 11/483,424

(22) Filed: Jul. 6, 2006

Related U.S. Application Data
(60) Provisional application No. 60/698,408, filed on Jul. 11, 2005.

(51) Int. Cl. A61B 17/22

(52) U.S. Cl. 606/160

(57) ABSTRACT

An apparatus is described including an elongate member having at least one finger positioned at a distal region of the elongate member. The finger, which can be detachable, is arranged and configured for cutting or scraping. In implementations including two or more fingers, distal ends of two or more fingers can be interconnected either directly or by both being connected to a distal tip of the elongate member.

### CROSS REFERENCE TO RELATED APPLICATION

This application claims priority to pending U.S. Provisional Application Ser. No. 60/698,408 entitled “Curette Heads”, filed on Jul. 11, 2005, the entire contents of which is hereby incorporated by reference.

### TECHNICAL FIELD

This invention relates to medical devices.

### BACKGROUND

When cancellous bone becomes diseased, for example, because of osteoporosis, avascular necrosis or cancer, the diseased bone may no longer provide adequate support to the surrounding cortical bone. The cortical bone may therefore become more prone to compression fracture or collapse.

The creation of cavities or voids within a structure (e.g., bone) in a subject can facilitate diagnostic or therapeutic intervention where disease is present. A curette is a surgical instrument used to remove tissue or growths from a body cavity and includes a curette head. The curette head can be shaped like a scoop or spoon to facilitate tissue removal.

### SUMMARY

This invention relates to medical devices. In general, in one aspect, the invention features an apparatus including an elongate member. The elongate member includes a first set of three or more fingers positioned at a distal region of the elongate member but proximal to a distal tip of the elongate member. Each finger includes a proximal and distal end and the distal ends of at least two of the fingers are connected to the distal tip of the elongate member. At least a portion of each of the fingers is configured for cutting or scraping.

Implementations of the invention can include one or more of the following features. The three or more fingers can be configured for cutting or scraping interior skeletal support structures of a subject selected from the group consisting of bone, cartilage and ossified derivatives thereof, membrane bone and cartilage bone. In one implementation, at least one finger is not connected at the finger's distal end to the distal tip of the elongate member.

The elongate member can be formed from a material selected from the group consisting of a metal, a shape memory material and a polymer. In one implementation the shape memory material is NITINOL.

At least one of the three or more fingers can include a cutting or scraping portion having a configuration selected from the group consisting of round coin-ended, rectangular coin-ended, curve-ended, multiple curve-ended, turn-ended, flattened coil-ended, flattened loop-ended, bent and coin-ended, coil-ended, bent coil-ended, hour glass coil-ended, osteotome-ended, whisk-ended, barb-ended, multiple curve-ended, hook-ended, sharp-ended, hair pin loop ended, bent-ended, press fit-ended, sickle ended, curved cannula-ended, crown-ended, mace-ended, helicopter ended, crisscross-ended, shovel-ended and multi-windowed tube-ended.

The three or more fingers can be deployable from a substantially collinear geometry to a substantially non-collinear geometry in relation to a longitudinal axis of the elongate member. The elongate member can further include a second set of three or more fingers positioned proximal the first set of three or more fingers, where each finger includes a proximal and distal end and the distal ends of at least two of the fingers are connected to the elongate member and where at least a portion of each of the fingers is configured for cutting or scraping. The second set of three or more fingers can be deployable from a substantially collinear geometry to a substantially non-collinear geometry in relation to a longitudinal axis of the elongate member.

In general, in another aspect, the invention features an apparatus including a cannula and an elongate member. The cannula includes an interior lumen and one or more apertures extending from the interior lumen to an exterior surface located in a distal portion of the cannula. The elongate member is positioned within the interior lumen of the cannula. The elongate member includes two or more fingers positioned at a distal region of the elongate member but proximal to a distal tip of the elongate member. Each finger includes a proximal and distal end and the distal end of at least one finger is connected to the distal tip of the elongate member. Each finger includes a cutting portion configured for cutting or scraping. The elongate member is positioned within the cannula such that the cutting portions of the fingers are deployable through the one or more apertures in the cannula.

Implementations of the invention can include one or more of the following features. The cannula distal portion can be configured to arrest movement of the distal tip of the elongate member. The cutting portions of the two or more fingers can be caused to deploy through the one or more apertures when the cannula distal portion arrests movement of the distal tip of the elongate member. The two or more fingers of the elongate member are comprised of a material selected from the group consisting of a metal, a shape memory material (e.g., NITINOL) and a polymer.

In one implementation, the distal portion of at least one of the fingers of the elongate member is not connected to the distal tip of the elongate member. The two or more fingers can be configured for cutting or scraping interior skeletal support structures of a subject selected from the group consisting of bone, cartilage and ossified derivatives thereof, membrane bone and cartilage bone.

In general, in another aspect, the invention features an apparatus including an elongate member. The elongate member is formed from a shape memory material and includes a set of two or more fingers positioned at a distal region of the elongate member but proximal to a distal tip of the elongate member. Each finger includes a proximal and distal end and the distal end of at least one of the fingers is connected to the distal tip of the elongate member and at least a portion of each of the fingers is configured for cutting or scraping.

Implementations of the invention can include one or more of the following features. In one implementation, the shape memory material is NITINOL. The two or more fingers can be formed of a material selected from the group consisting of a metal, a shape memory material (e.g., NITINOL) and a polymer. The two or more fingers can be detachable from the elongate member.

In one implementation, one or more of the fingers includes a cutting or scraping portion having a configuration selected from the group consisting of round coin-ended, rectangular coin-ended, curve-ended, multiple curve-ended, turn-ended, flattened coil-ended, flattened loop-ended, bent and coin-ended, coil-ended, bent coil-ended, hour glass coil-ended, osteotome-ended, whisk-ended, barb-ended, multiple curve-ended, hook-ended, sharp-ended, hair pin loop ended, bent-ended, press fit-ended, sickle ended, curved cannula-ended, crown-ended, mace-ended, helicopter ended, crisscross-ended, shovel-ended and multi-windowed tube-ended.

The two or more fingers can be deployable from a substantially collinear geometry to a substantially non-collinear geometry in relation to a longitudinal axis of the elongate member. The two or more fingers can be configured for cutting or scraping interior skeletal support structures of a subject selected from the group consisting of bone, cartilage and ossified derivatives thereof, membrane bone and cartilage bone.

Aspects of the invention may include one or more of the following advantageous features. The elongate member can include a single piece design, providing simplicity, reduced size and strength. Reducing the size of the elongate member and the fingers facilitates cutting or scraping of smaller structures in a subject.

In addition to cutting or scraping, the fingers of the elongate member can serve to capture and remove portions of structures being manipulated. For example, where the fingers cut or scrape a structure in a partial or fully deployed configuration, the fingers can capture the resulting fragments or portions of structures for removal by grasping or closing upon the fragments. Once captured, the fragments or portions can be drawn away from the structure being manipulated.

The resilient spring-like properties of the elongate member can facilitate cutting or scraping. For example, when the elongate member is advanced into contact with a structure in a subject, the spring-like properties can cause the elongate member to remain in contact with the structure. Additionally, the resilient nature of the elongate member can facilitate passage of a compressed elongate member through a cannula.

The fingers and/or the entire elongate member can include NITINOL having a variety of activation temperatures depending on user need. For example, the NITINOL can include activation temperatures of, for example, room temperature or body temperature (37° C.). When superelastic NITINOL is used the fingers of the elongate member can be shape-set in a desired configuration for cutting or scraping that represents the apparatus native conformation. When a cannula or delivery tube is pushed over the distal end, the finger-shape is collapsed, facilitating delivery to a structure in a subject. As the cannula or delivery tube is pulled back to reveal the fingers, they expand to the native conformation.

When the elongate member is used for cutting or scraping a skeletal support structure, for example, within a vertebral body, the cancellous bone can be scored without damaging the vertebral body endplates.

The elongate member can include a lumen, wherein the lumen serves as a conduit or passageway for substances or devices including, but not limited to, needles, curettes, catheters, balloon-catheters and the likes.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A shows an apparatus including an elongate member having fingers.

FIG. 1B shows an apparatus including an elongate member having multiple sets of fingers.

FIG. 1C shows an apparatus including a cannula and an elongate member having fingers.

FIG. 1D shows an apparatus including an elongate member having fingers including both free and interconnected distal portions.

FIG. 1E shows an apparatus including an elongate member having fingers without interconnected distal portions.

FIGS. 2A-2II shows fingers including various cutting or scraping portions.

FIG. 3A shows an elongate member including a coupler and fingers.

FIG. 3B shows a coupler.

FIG. 3C shows an elongate member including a coupler and fingers.

FIG. 4A shows an apparatus including a cannula and an elongate member including a finger.

FIG. 4B shows a cannula including an interior lumen.

FIG. 4C shows a cannula including apertures.

FIG. 4D shows an apparatus including a cannula having apertures and an elongate member in a collapsed configuration.

FIG. 4E shows an apparatus including a cannula having apertures and an elongate member in a deployed configuration.

FIG. 5A shows an apparatus including a cannula and an elongate member including fingers in a first partially deployed configuration.

FIG. 5B shows an apparatus including a cannula and an elongate member including fingers in a second partially deployed configuration.

FIG. 5C shows an apparatus including a cannula and an elongate member including fingers in a third partially deployed configuration.

FIG. 5D shows an apparatus including a cannula and an elongate member including fingers in a fully deployed configuration.

Like reference symbols in the various drawings indicate like elements.

### DETAILED DESCRIPTION

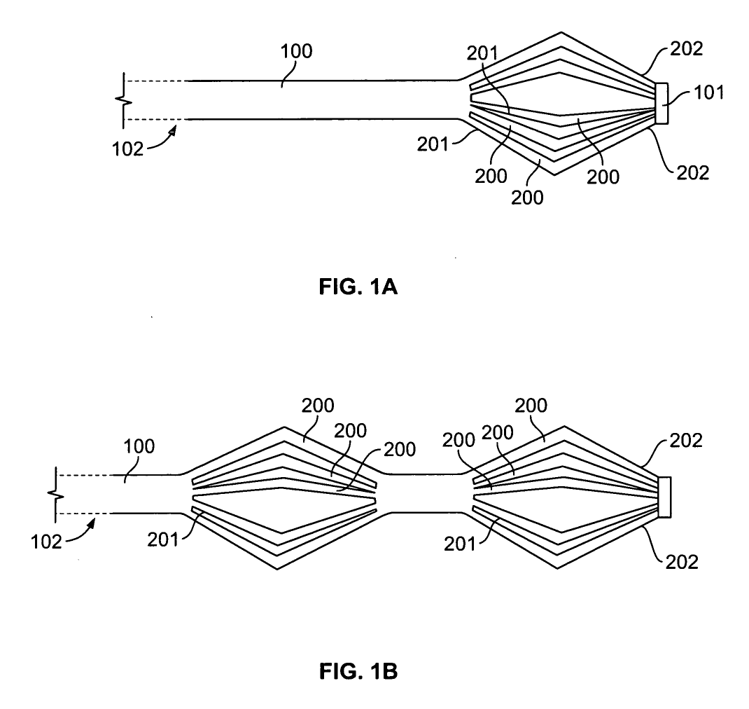

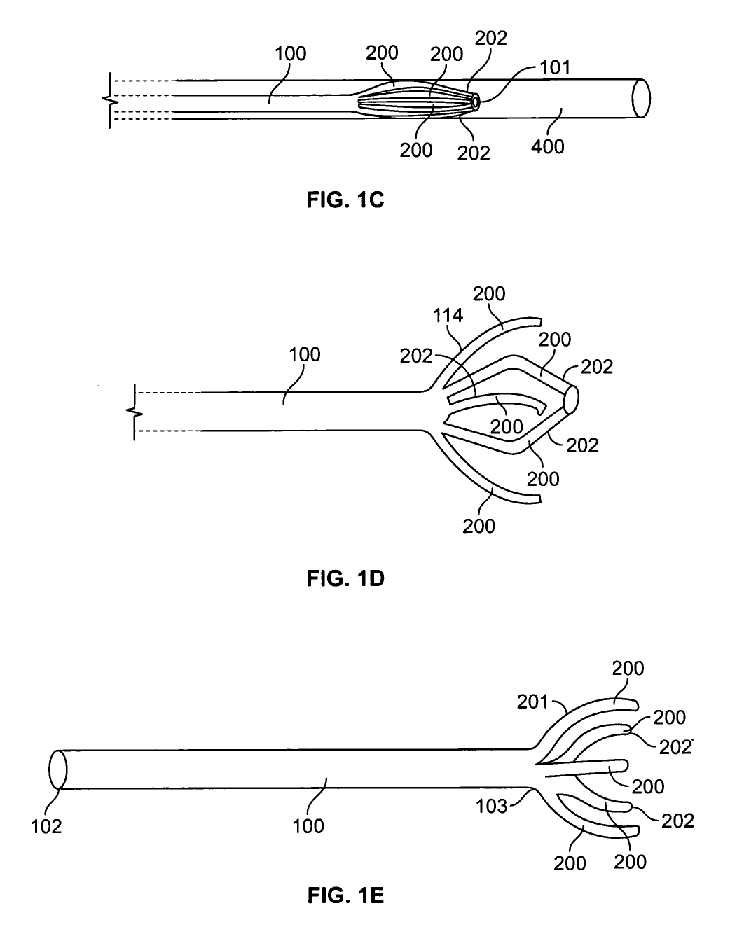

FIGS. 1A-E, 4A, 4D-F, and 5A-D show an apparatus including an elongate member 100 for cutting or scraping structures (e.g., interior skeletal support structures) of a subject. As shown in FIG. 1A-B, 1E, 4A, and 4D-E, the elongate member 100 includes a proximal portion 102, a distal portion 101 and fingers 200. The elongated member 100 can be configured, for example, as a curette, wire, pick, needle or other suitable cutting or scraping device. Cutting or scraping structures of a subject can include, but are not limited to, the cutting and scraping of bone, cartilage and ossified derivatives thereof, membrane bone and cartilage bone.

As shown in FIGS. 1A-E, 4A, 4D-E, 3A, 3C, and 5A-D, in particular implementations the elongate member 100 includes three or more fingers 200 having proximal ends 201 and distal end 202. In one implementation, the fingers 200 are detachable. The fingers' proximal ends 201 are connected to a distal region of the elongate member distal portion 101. The fingers 200 are arranged and configured for cutting or scraping structures of a subject. Additionally, the distal ends 202 of two or more fingers 200 are connected to a distal tip 101 of the elongate member 100 and are thereby interconnected. In the implementation shown in FIG. 1B, two sets of fingers 200 can be arranged within the distal region of the elongate member 100 in tandem.

In the implementation shown in FIGS. 1A-C, 3A and 4D-E, each finger 200 of the elongate member 100 is interconnected at the finger distal end 202 to one or more other finger distal end 202, as all distal ends are connected to the distal tip 101 of the elongate member 100. In another implementation, the distal end 202 of each finger 200 can be interconnected by common attachment to, for example, a ring, disc, plug, tube or other suitable attachment point. As shown in FIG. 1D, in another implementation the elongate member 100 can include a combination of two or more interconnected fingers 200 (i.e., fingers 200 connected at distal ends 202) and one or more fingers where the finger's distal end 202 remains free in relation to other finger distal ends 202.

In one implementation elongate member 100 is formed from a material such as a metal, a shape memory material or a polymer. A metal can include, for example, cobalt-chrome (L605), ASTMf 90, 304/216 spring tempered stainless steel, titanium, and nickel-titanium. A shape memory material can include, for example, NITINOL (an acronym for NIckel TItanium Naval Ordnance Laboratory), a family of intermetallic materials, which contain a nearly equal mixture of nickel (55 wt. %) and titanium. In another implementation, other elements can be added to NITINOL to adjust or “tune” the material properties. A polymer can include, for example, polycarbonate or nylon (glass-filled).

The elongate member 100 can include a lumen extending from proximal portion 102 to the distal region. In one implementation the lumen is a feature of the elongate member 100 where elongate member 100 is formed in a tube configuration.

As shown in FIG. 1E, in a particular implementation the apparatus includes the elongate member 100 which is formed from a shape memory material. The elongate member 100 includes a proximal portion 102, distal region 103 and two or more deployable fingers 200, each finger including a proximal end 201 and a distal end 202. The proximal ends 201 of the fingers 200 are connected to the distal region 103 of the elongate member and the fingers 200 are configured for cutting or scraping.

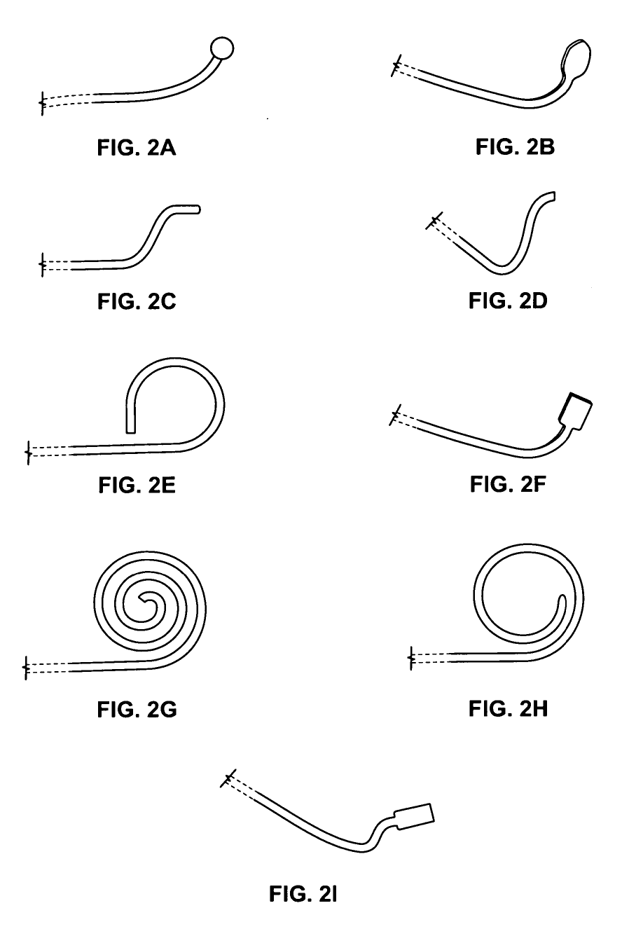

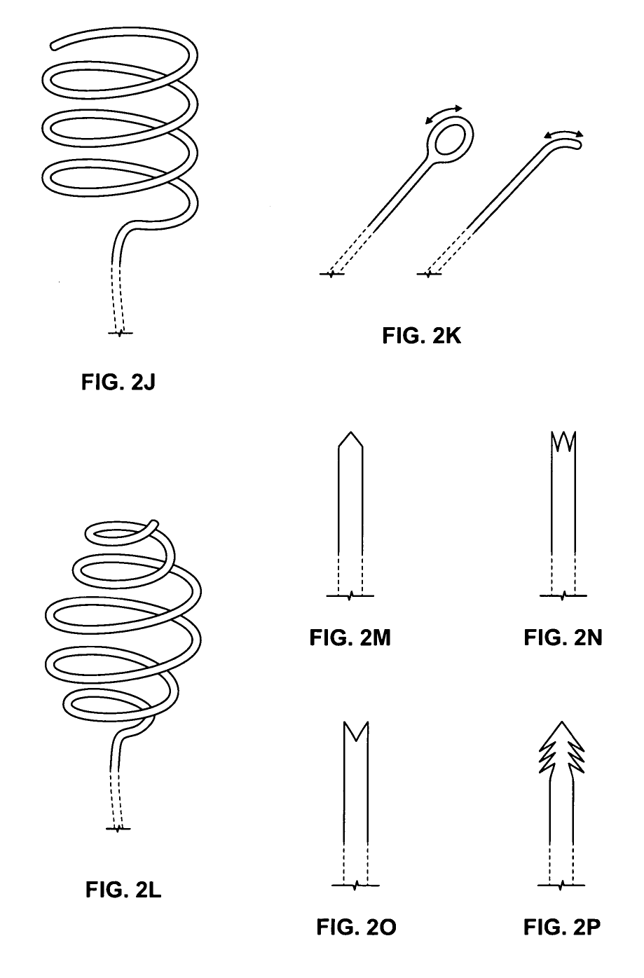

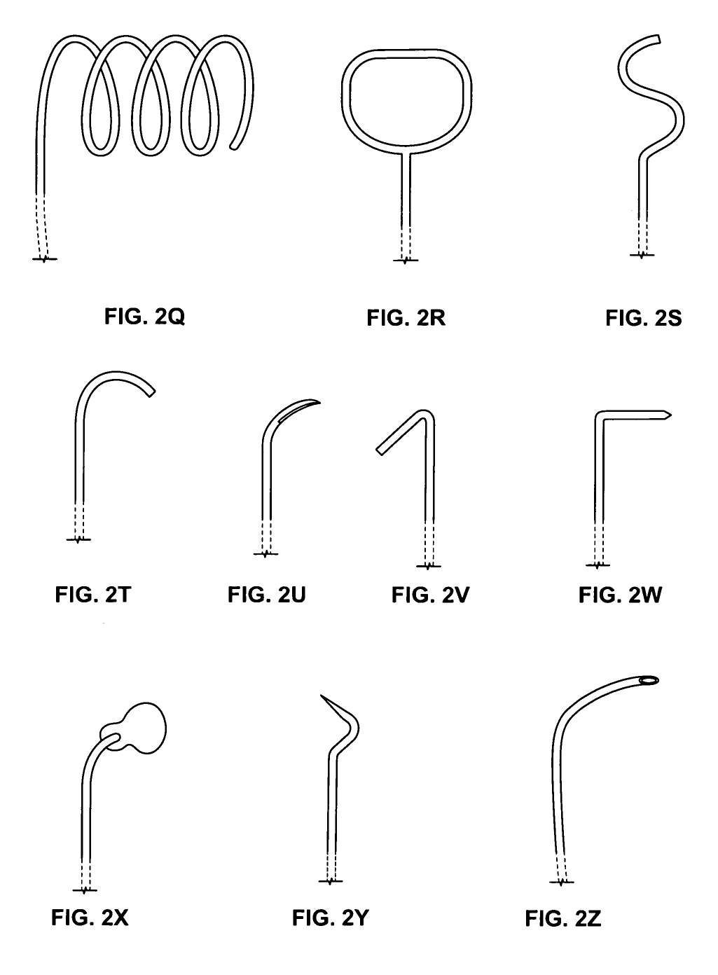

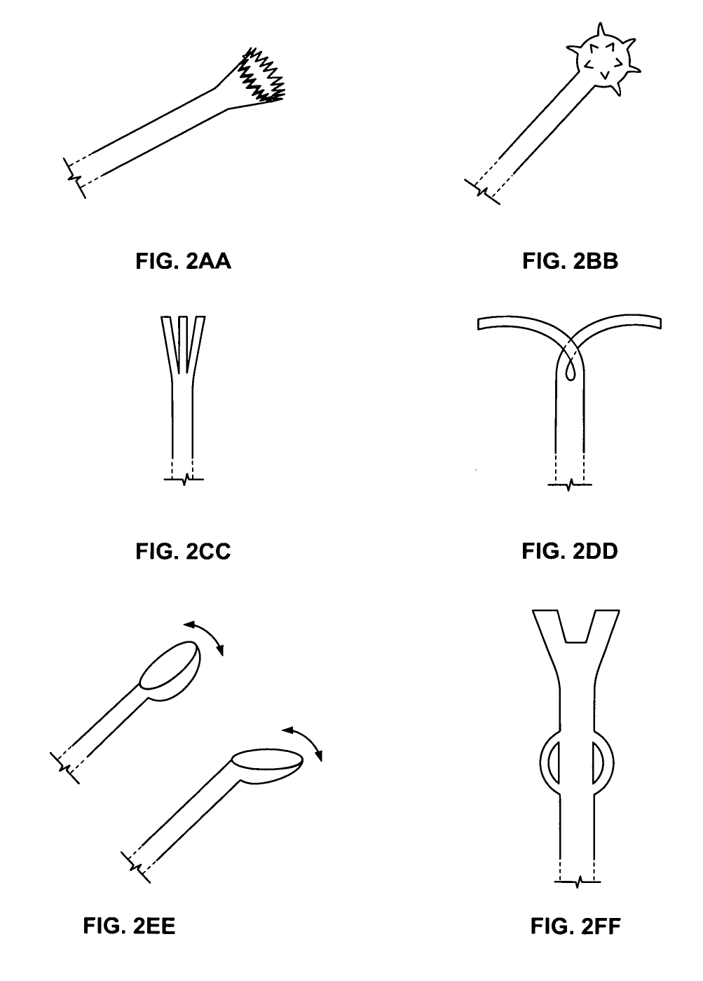

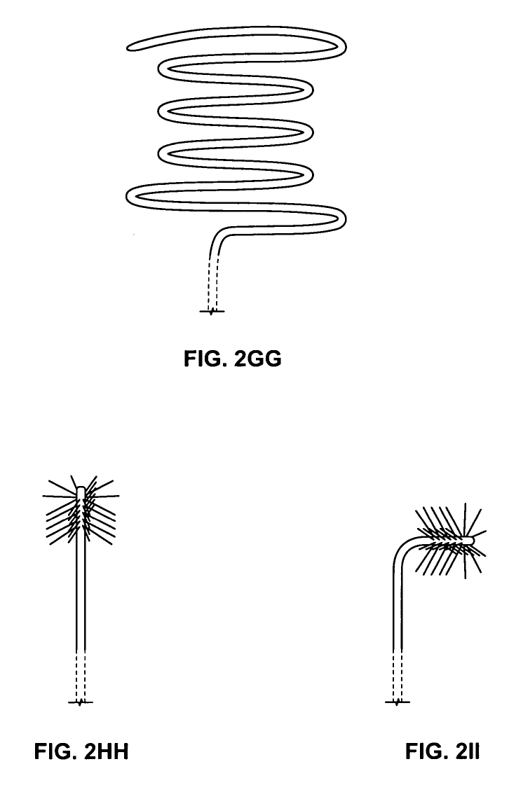

As shown in FIGS. 2A-II, the fingers 200 can include a cutting or scraping portion. Examples of suitable cutting or scraping portions include but are not limited to ball-ended (see FIG. 2A), coin-ended (see FIG. 2B), curve-ended (see FIG. 2C), turn-ended (see FIG. 2D), docking-ended (see FIG. 2E), square coin-ended (see FIG. 2F), flattened coil-ended (see FIG. 2G), flattened loop ended (see FIG. 2H), bent and coined-ended (see FIG. 21), coil-ended (see FIG. 2J), osteotome-ended (see FIG. 2K), whisk-ended (see FIG. 2L), barb-ended (see FIGS. 2M-P), bent coil-ended (see FIG. 2Q), loop-ended, (see FIG. 2R), multiple curve-ended (see FIG. 2S), hook-ended (see FIG. 2T), sharp-ended (see FIG. 2U), hair pin loop ended (see FIG. 2V), bent-ended (see FIG. 2W), press fit-ended (see FIG. 2X), sickle ended (see FIG. 2Y), curved cannula-ended (see FIG. 2Z), crown-ended (see FIG. 2AA), mace-ended (see FIG. 2BB), helicopter-ended (see FIG. 2CC), crisscross-ended (see FIG. 2DD), shovel-ended (see FIG. 2EE), multi-windowed tube-ended (see FIG. 2FF), hourglass coil-ended (see FIG. 2GG), brush-ended (see FIG. 2HH) and bent brush-ended (see FIG. 2II).

Actuation of cutting or scraping with the fingers 200 can be achieved, for example, through a forward and back flexing movement of fingers 200 in relation to the elongate member 100. Such a movement can be driven by a drive mechanism (e.g., hydraulic). As shown in FIG. 2K, where the finger 200 cutting or scraping portion is osteotome-ended, the finger 200 can be formed from, for example, nickel-titanium and the osteotome end can be actuated in a forward and back movement.

As shown in FIG. 2CC, where the finger 200 cutting or scraping portion is helicopter-ended, the actuation of cutting or scraping can include interconversion of the finger 200 from a low profile folded configuration to an unfolded configuration.

As shown in FIG. 2EE, where the finger 200 cutting or scraping portion is shovel-ended, the activation of cutting or scraping can include a scooping and dumping series of motions. Other cutting or scraping portions of fingers 200 can include needle-ended, bone chisel-ended and safety wire-ended (braided wire-ended).

In use, actuating cutting or scraping using the fingers 200 can include impacting a finger 200 cutting or scraping portion upon a structure in a subject. Impacting the structure can be achieved using a chiseling, jack hammering motion or twisting motion.

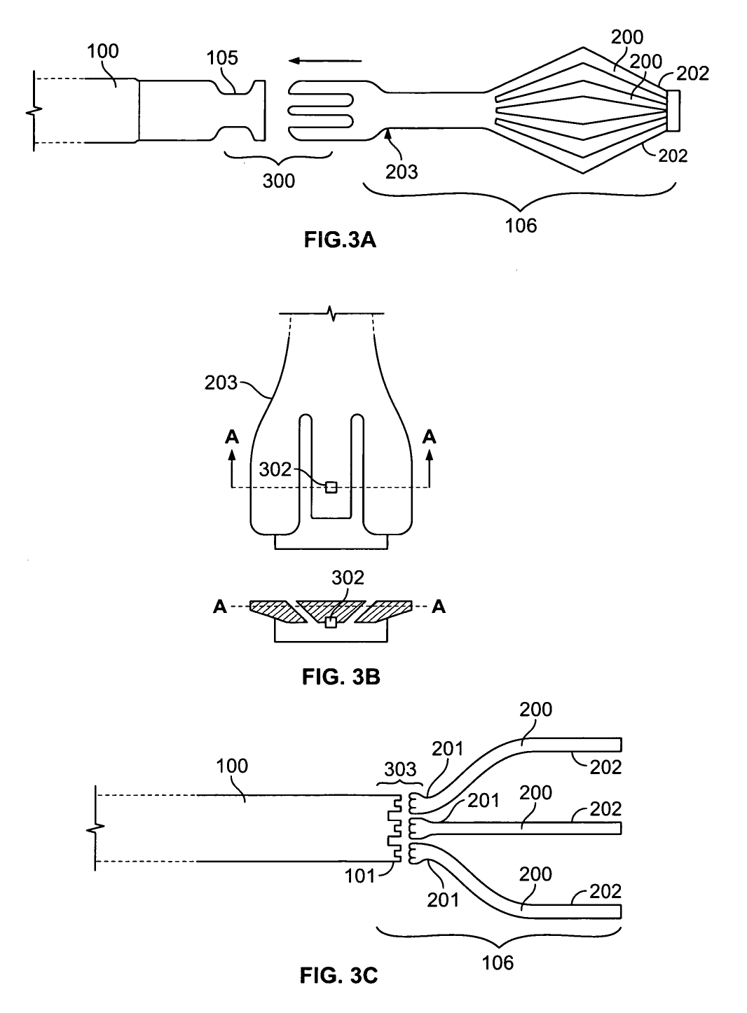

As shown in FIGS. 3A-C, in one implementation, the fingers 200 are detachable from the elongate member 100. FIG. 3A shows one implementation where multiple fingers 200 can be interconnected to elongate member 100 as a unit using a coupler 300. The coupler 300 includes a shaped elongate member distal end 105 and a complementary-shaped finger proximal portion 203. In this implementation, the shape of elongate member distal end 105 and the complementary shape of finger proximal portion 205 can be any of a number of configurations including, but not limited to, snap-in, clip-in, press-fit or other suitable detachable interconnection.

As shown in FIG. 3B, the coupler 300 can include a detent 302 integrated into the finger proximal portion 203, and a complementary protrusion 301 extending from elongate member distal end 105. In use, the detent 302 and protrusion 301 can reversibly interconnect when the elongate member distal end 105 is caused to engage the finger proximal portion 203. Alternatively, in another implementation, the detent 302 is integrated into the elongate member distal end 105 and the protrusion 401 extends from the finger proximal portion 203.

FIG. 3C show another implementation where individual fingers 200 can be interconnected to the elongate member distal end 105 using individual couplers 303. In this implementation, each individual coupler 303 also includes an elongate member distal end 107 and a complimentary-shaped finger proximal portion 207. Each coupler 303 can be configured similar to the couple 300 shown in FIG. 3B.

In another implementation, the couplers 300 or 303 include a threaded interconnection between the finger proximal portion 203 or 207 and the elongate member distal end 203 or 207. For example, a threaded nickel-titanium finger proximal portion 201 can be screwed onto a distal end 105 or 107 of a threaded stainless steel elongate member 100.

In another implementation, elongate member distal end 105 or 107 can include a keyway into which the finger proximal portion 203 or 207 can be interconnected. The elongate member distal end 105 or 107 can further include external threads and a threaded locking means for securing one or more fingers 200 to the elongate member 100.

In further implementations couplers 300 or 303 can include an interconnection arrangement including, for example, crush-pins, snap-fittings, leaf springs, magnetic hex-tips, quick connects, ball detents or crimps.

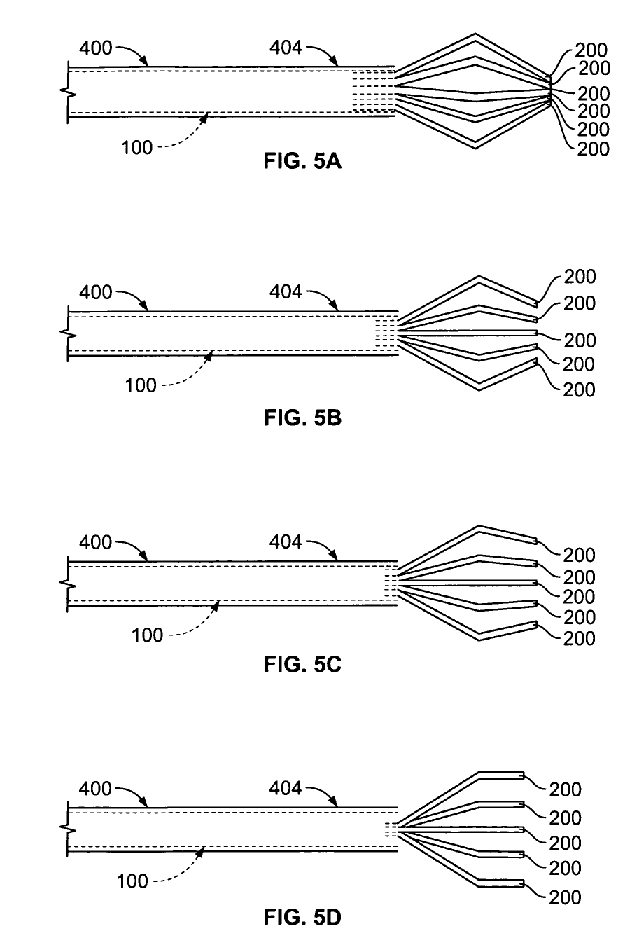

As shown in FIGS. 1A and C, in one implementation the fingers 200 of elongate member 100 are deployable from a substantially collinear geometry (see FIG. 1C) to a substantially non-collinear geometry (see FIG. 1A) in relation to the longitudinal axis of elongate member 100. Additionally, as shown in FIGS. 5A-D, in another implementation, the fingers 200 of the elongate member 100 are deployable from a substantially collinear geometry (see FIG. 5A) progressively to a substantially non-collinear geometry (see FIG. 5B-D) in relation to the longitudinal axis of elongate member 100. In the implementation shown in FIGS. 5A-D, a cannula 400 is used to govern the progress of fingers 200 deployment, based on the elastic nature of fingers 200 and the degree to which the cannula 400 encloses the fingers 200. As shown in FIGS. 5A-D, as an increased length of the fingers 200 length is exposed from the cannula 400, the fingers 200 progressively deploy until maximum deployment occurs (see FIG. 5D).

In use, after accessing a structure, the cutting or scraping portions of fingers 200 can be used to create a void within the structure. As used herein, “create a void” is meant to include both expanding an existing void in a skeletal support structure in addition to expanding the interior of a skeletal support structure to produce a void. It is contemplated that a skeletal support structure accessed with the elongate member 100 can include a void prior to being accessed or upon being accessed. It is further contemplated that such a prior existing or contemporaneously formed void can be further expanded using the elongate member 100. A void can be created in a skeletal support structure using the elongate member 100, for example, wherein a user employs the elongate member 100 in cutting, scrapping, tamping, drilling or other suitable manipulations for engaging the skeletal support structure.

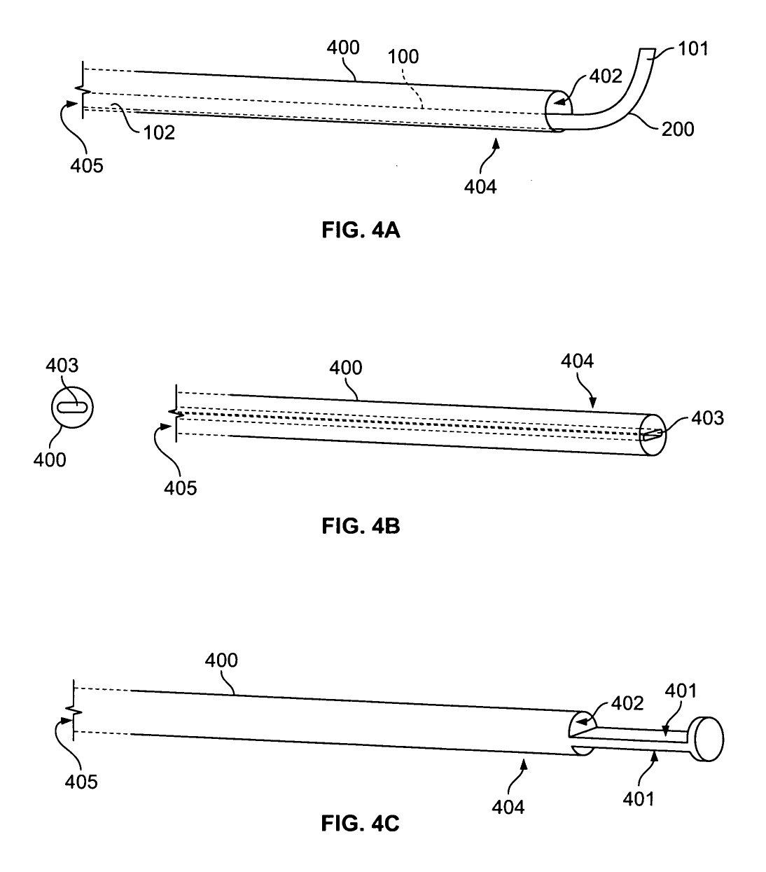

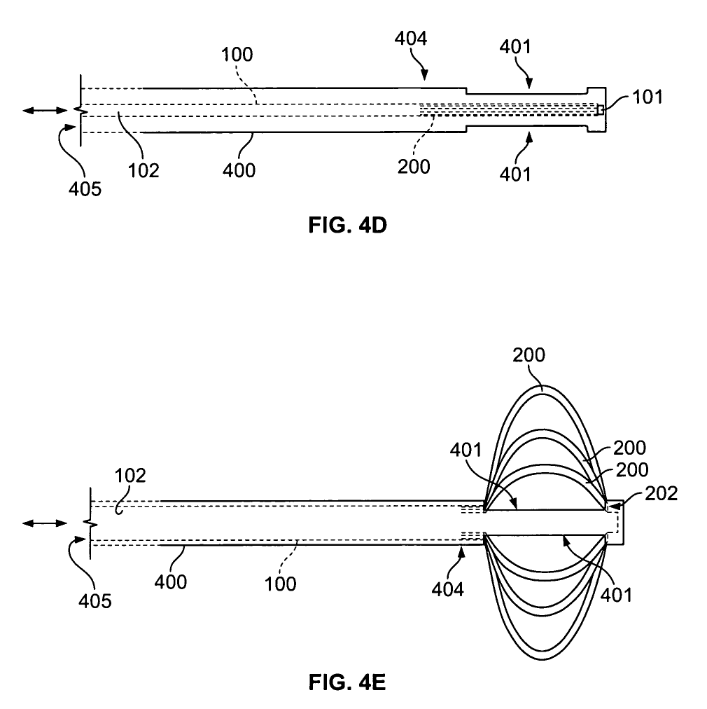

As shown in FIGS. 4A-E, any of a number of cannulas 400 can be used in conjunction with elongate member 100. Useful cannulas 400 can include but are not limited to, a tubular cannula 400 (see FIG. 4A), a cannula 400 having an oblong cross-section interior lumen 403 (see FIG. 4B), or a cannula 400 having one or more apertures 401 located in the cannula distal portion 404 (see FIGS. 4C-E). As shown in FIGS. 1C and 4D, a cannula 400 can be useful for delivering the elongate member 100 to a structure, where the fingers 200 of elongate member 100 are maintained in an un-deployed configuration when contained within the cannula 400. Deployment of elongate member fingers 200 can be accomplished, for example, as discussed in the following implementations.

As shown in FIGS. 4A and 5A-D, when the elongate member 100 is advanced beyond a distal portion 404 of cannula 400, the fingers 200 can deploy. As shown in FIGS. 5A-D, positioning the elongate member 100 within cannula 400 can be used to control deployment of the fingers 200. Deployment can be incrementally regulated by positioning the elongate member 100 within cannula 400, to provide degrees of partial deployment (see FIGS. 5A-C) or full deployment (see FIG. 5D).

Deployment of the fingers 200 can result from inherent properties associated with materials from which the fingers 200 are constructed. For example, where the fingers 200 are constructed of a metal, the fingers 200 can deploy to a given pre-formed shape as a result of the spring-like nature of the metal. Alternatively, wherein the fingers 200 are constructed from a shape-memory material (e.g. NITINOL) the deployment of fingers 200 can be regulated using temperature variation.

As shown in FIG. 4B, in one implementation, the cannula 400 includes an oblong cross-section interior lumen 403. When such a cannula 400 is used in combination with an elongate member 100 having a complementary geometry, the interior lumen 403 can function to orientate movement of the elongate member 100 in a plane, such that in use, cutting or scraping with elongate member 100 in a given plane can be controlled.

As shown in FIGS. 4C-E, in one implementation, the cannula 400 includes a proximal portion 405, a distal portion 404 and one or more apertures 401. The apertures 401 provide an egress and re-entry route for the fingers 200 of elongate member 100 from the cannula interior lumen 402. The cannula 400 can include any number of apertures 401, for example, a single aperture 401 or two or more apertures 401. The apertures 401 can be arranged in any of a number of configurations, including but not limited to slot(s), hole(s), or the like. As shown in FIGS. 4D-E, a combination of the elongate member 100 with the cannula 400, including the one or more apertures 401, can be configured and arranged for delivering and deploying the elongate member 100 to a structure. As shown in FIGS. 4D-E, in one implementation of such a combination, the elongate member 100 includes the proximal portion 102, the distal tip 101 and two or more fingers 200 arranged and configured for cutting or scraping.

As shown in FIGS. 4D-E, in a particular implementation where the apparatus includes the combination of the elongate member 100 and a cannula 400, the cannula's distal portion 404 is arranged and configured to arrest movement of the elongate member's distal tip 101. As shown in FIG. 4E, after elongate member distal portion tip 101 is arrested, cutting portions of two or more fingers 200 can be caused to deploy through one or more apertures 401. In use, deployment is achieved when the elongate member 100 is advanced to cannula distal portion 404 until movement is arrested (see FIG. 4D). Subsequently, as shown in FIG. 4E, further advancement of elongate member 100 results in deployment of elongate member fingers 200 through one or more apertures 401. The amount of advancement of elongate member 100 within cannula 400 can be used to control deployment of fingers 200. Deployment can be incrementally regulated by positioning elongate member 100 within cannula 400, to provide degrees of partial deployment (not shown) or full deployment (see FIG. 4E). The deployment process for fingers 200 can be reversed, for example, by drawing the elongate member's distal tip 101 away from the cannula distal portion 404.

In the preceding implementation, the two or more fingers 200 can be formed of a material including but not limited to a metal, a shape memory material or a polymer. In a particular implementation, the shape memory material is NITINOL. Additionally, the distal ends 202 of two or more of the fingers 200 can be interconnected to one or more other finger distal end 202. For example, the distal ends 202 of two fingers 200 can be interconnected, either directly or by both being connected to the distal tip 101 of the elongate member. Similarly, the distal ends 202 of three or more fingers 200 can be interconnected (see FIGS. 4D-E). Alternatively, where two fingers 200 are interconnected and a third or more additional finger(s) 200 are included in elongate member 100, the distal ends 202 of the additional finger(s) 200 can be free from connection to any other finger(s) 200. It is envisioned that any of a number of combinations of interconnected and unconnected fingers 200 can be included in the elongate member 100. In one implementation a minimum of two fingers 200 are interconnected and/or both connected at their distal ends 202 to the distal tip 101 of the elongate member 100.

In use, the above described elongate member 100 can be used to create a void in a skeletal support structure using elongate member 100, for example, wherein a user employs the elongate member 100 in cutting, scrapping, tamping, drilling or other suitable manipulations for engaging the skeletal support structure.

A number of embodiments of the invention have been described. Nevertheless, it will be understood that various modifications may be made without departing from the spirit and scope of the invention. Accordingly, other embodiments are within the scope of the following claims.

What is claimed is:

1. An apparatus comprising:
an elongate member including a first set of three or more fingers positioned at a distal region of the elongate member but proximal to a distal tip of the elongate member, where each finger includes a proximal and distal end and the distal ends of at least two of the fingers are connected to the distal tip of the elongate member and where at least a portion of each of the fingers is configured for cutting or scraping.

2. The apparatus of claim 1, where the three or more fingers are configured for cutting or scraping interior skeletal support structures of a subject selected from the group consisting of bone, cartilage and ossified derivatives thereof, membrane bone and cartilage bone.

3. The apparatus of claim 1, where at least one finger is not connected at the finger's distal end to the distal tip of the elongate member.

4. The apparatus of claim 1, where the elongate member is comprised of a material selected from the group consisting of a metal, a shape memory material and a polymer.

5. The apparatus of claim 4, where the shape memory material is NITINOL.

6. The apparatus of claim 1, where at least one of the three or more fingers comprise a cutting or scraping portion having a configuration selected from the group consisting of round coin-ended, rectangular coin-ended, curve-ended, multiple curve-ended, turn-ended, flattened coil-ended, flattened loop-ended, bent and coin-ended, coil-ended, bent coil-ended, hour glass coil-ended, osteotome-ended, whisk-ended, barb-ended, multiple curve-ended, hook-ended, sharp-ended, hair pin loop ended, bent-ended, press fit-ended, sickle ended, curved cannula-ended, crown-ended, mace-ended, helicopter ended, crisscross-ended, shovel-ended and multi-windowed tube-ended.

7. The apparatus of claim 1, where the three or more fingers are deployable from a substantially collinear geometry to a substantially non-collinear geometry in relation to a longitudinal axis of the elongate member.

8. The apparatus of claim 1, the elongate member further comprising:
a second set of three or more fingers positioned proximal the first set of three or more fingers, where each finger includes a proximal and distal end and the distal ends of at least two of the fingers are connected to the elongate member and where at least a portion of each of the fingers is configured for cutting or scraping.

9. The apparatus of claim 8, where the second set of three or more fingers are deployable from a substantially collinear geometry to a substantially non-collinear geometry in relation to a longitudinal axis of the elongate member.

10. An apparatus comprising:
a cannula including an interior lumen and one or more apertures extending from the interior lumen to an exterior surface located in a distal portion of the cannula;
an elongate member positioned within the interior lumen of the cannula, the elongate member including:
two or more fingers positioned at a distal region of the elongate member but proximal to a distal tip of the elongate member, where each finger includes a proximal and distal end and the distal end of at least one finger is connected to the distal tip of the elongate member and where each finger includes a cutting portion configured for cutting or scraping;
where the elongate member is positioned within the cannula such that the cutting portions of the fingers are deployable through the one or more apertures in the cannula.

11. The apparatus of claim 10, where the cannula distal portion is configured to arrest movement of the distal tip of the elongate member.

12. The apparatus of claim 11, where the cutting portions of the two or more fingers are caused to deploy through the one or more apertures when the cannula distal portion arrests movement of the distal tip of the elongate member.

13. The apparatus of claim 10, where the two or more fingers of the elongate member are comprised of a material selected from the group consisting of a metal, a shape memory material and a polymer.

14. The apparatus of claim 13, where the shape memory material is NITINOL.

15. The apparatus of claim 10, where the distal portion of at least one of the fingers of the elongate member is not connected to the distal tip of the elongate member.

16. The apparatus of claim 10, where the two or more fingers are configured for cutting or scraping interior skeletal support structures of a subject selected from the group consisting of bone, cartilage and ossified derivatives thereof, membrane bone and cartilage bone.

17. An apparatus comprising:
an elongate member formed from a shape memory material and including a set of two or more fingers positioned at a distal region of the elongate member but proximal to a distal tip of the elongate member, where each finger includes a proximal and distal end and the distal end of at least one of the fingers is connected to the distal tip of the elongate member and where at least a portion of each of the fingers is configured for cutting or scraping.

18. The apparatus of claim 17, where the shape memory material is NITINOL.

19. The apparatus of claim 17, where the two or more fingers are comprised of a material selected from the group consisting of a metal, a shape memory material and a polymer.

20. The apparatus of claim 19, where the shape memory material is NITINOL.

21. The apparatus of claim 17, where the two or more fingers are detachable from the elongate member.

22. The apparatus of claim 17, where one or more of the fingers comprise a cutting or scraping portion having a configuration selected from the group consisting of round coin-ended, rectangular coin-ended, curve-ended, multiple curve-ended, turn-ended, flattened coil-ended, flattened loop-ended, bent and coin-ended, coil-ended, bent coil-ended, hour glass coil-ended, osteotome-ended, whisk-ended, barb-ended, multiple curve-ended, hook-ended, sharp-ended, hair pin loop ended, bent-ended, press fit-ended, sickle ended, curved cannula-ended, crown-ended, mace-ended, helicopter ended, crisscross-ended, shovel-ended and multi-windowed tube-ended.

23. The apparatus of claim 17, where the two or more fingers are deployable from a substantially collinear geometry to a substantially non-collinear geometry in relation to a longitudinal axis of the elongate member.

24. The apparatus of claim 17, where the two or more fingers are configured for cutting or scraping interior skeletal support structures of a subject selected from the group consisting of bone, cartilage and ossified derivatives thereof, membrane bone and cartilage bone.
